(19) United States
(12) Patent Application Publication
(10) Pub. No.: US 2019/0192218 A1
(43) Pub. Date: Jun. 27, 2019

(54) SURGICAL DEVICE AND METHOD OF USE

(71) Applicant: Hermes Innovations, LLC, Cupertino, CA (US)

(72) Inventors: Benedek Orczy-Timko, Budapest (HU); Csaba Truckai, Saratoga, CA (US)

(73) Assignee: Hermes Innovations, LLC, Cupertino, CA (US)

(21) Appl. No.: 16/222,757

(22) Filed: Dec. 17, 2018

Related U.S. Application Data
(63) Continuation of application No. 14/864,379, filed on Sep. 24, 2015, now abandoned.
(60) Provisional application No. 62/058,277, filed on Oct. 1, 2014.

(51) Int. Cl. A61B 18/14; A61B 17/32

(52) U.S. Cl. CPC A61B 18/1485; A61B 2017/3454; A61B 2217/005; A61B 17/32002

(57) ABSTRACT

A tissue resecting device includes a handle coupled to an elongated sleeve assembly. The elongated sleeve assembly includes a windowed outer sleeve and an inner sleeve adapted to reciprocate and/or rotate relative to the window to resect tissue intruding into the window, where the resected tissue is captured in a channel in the sleeve assembly. One or more motors in the handle both move the inner sleeve relative to the window and operate a pump which causes a fluid to flow through the channel in the sleeve assembly to remove resected tissue therefrom.

### CROSS-REFERENCE TO RELATED APPLICATIONS

This application is a continuation of U.S. patent application Ser. No. 14/864,379 (Attorney Docket No. 37644-710.201), filed Sep. 24, 2015, which claims the benefit of U.S. Provisional Application No. 62/058,277 (Attorney Docket No. 37644-710.101), filed Oct. 1, 2014, the entire contents of which is incorporated herein by reference.

### BACKGROUND OF THE INVENTION

### 1. Field of the Invention

The present invention relates to devices and methods for resecting and removing tissue from the interior of a patient's body, for example in a transurethral resection of prostate tissue to treat benign prostatic hyperplasia.

### 2. Description of the Background Art

Transurethral resection of the prostate (TURP) typically relies on insertion of an instrument through the urethra to remove a section of the prostate that is blocking urine flow. The instrument may take a variety of forms, often using radio frequency (RF) loops or blades that are drawn across an inner wall of the urethra within the prostate to “debulk” the prostate tissue.

While generally effective, many prior electrosurgical TURP and other prostate resection devices have difficulty in remove resected tissue from the urethra during and after treatment.

For these reasons, it would be desirable to provide improved devices and methods for performing TURP and other tissue resection procedures. It would be particularly desirable if such devices and methods provided alternative and more effective structures and procedures for removing resected tissue during and after a resection procedure. At least some of these objectives will be met by the inventions described below.

### BRIEF SUMMARY OF THE INVENTION

In a first aspect of the present invention, a tissue resecting device includes handle coupled to an elongated sleeve assembly. The elongated sleeve assembly includes a windowed outer sleeve and an inner sleeve adapted to move relative to the window to resect tissue intruding into the window. The resected tissue is captured in a channel in the sleeve assembly, and at least one motor in the handle is configured to both move the inner sleeve relative to the window and to operate a pump which causes a fluid to flow through the channel in the sleeve assembly to remove resected tissue therefrom.

In some embodiments, a single motor is configured and connected both to move the inner sleeve and to operate the pump. In other embodiments, a first motor moves the inner sleeve and a second motor operates the pump. The motor or motors are usually electric but could also be pneumatic or hydraulic.

The inner and outer sleeves may be configured in a variety of ways. Often the sleeves are mounted coaxially, and the inner sleeve is adapted to reciprocate relative to the outer sleeve and the window. Alternatively or additionally, the inner sleeve may be adapted to rotate relative to the window. The inner sleeve will usually have a cutting blade or edge, typically being a sharpened blade or an electrosurgical edge, e.g. being an electrode for electrosurgically resecting tissue. The blade or cutting edge can be oriented in a variety of ways. The edge/blade can comprise the forward circular tip of the inner sleeve when the inner sleeve is to be reciprocated, either alone or in combination with rotation or oscillation. The edge or blade may be oriented along an axial line when the inner sleeve is to be rotated, optionally in combination with reciprocation. A number of other orientations, such as angled or irregular, would also be possible.

The pump(s) is usually a positive displacement pump, for example comprising any one of a piston pump, a screw pump, an impeller pump, a peristaltic pump, a vane pump, a lobe pumps, a diaphragm pump, or the like. The pump typically provides a fluid flow, often pulsed, to an open termination in a distal tip portion of the channel from where it can flow back through the channel to create a positive pressure to push resected tissue out through the channel. Usually, a vacuum will simultaneous and/or sequentially applied at a proximal end of the channel to further cause the tissue to be withdrawn from the channel in a proximal direction.

In a second aspect of the present invention, a tissue resecting system includes a handle coupled to an elongated sleeve assembly comprising a windowed outer sleeve and an inner sleeve. The inner sleeve is adapted to move in a cycle to resect tissue protruding into the window and deposit the tissue in a channel in the sleeve assembly, and a pump mechanism in or proximate the handle is configured to provide a fluid flow through the to expel the resected tissue from the channel.

The pump mechanism may be adapted to provide flow at a constant rate over each cycle of the inner sleeve. Alternatively, the pump mechanism may be adapted to provide the flow at a non-constant rate over each cycle of the inner sleeve. Typically, the pump mechanism is adapted to provide the flow in at least one pulse or a series of discrete pulses. Often, the pump mechanism is adapted to provide flow primarily or only when the inner sleeve is positioned to cover or close the window in the outer sleeve.

The pump mechanism will usually provide a flow volume in a range between 1 cc and 10 cc during each cycle of the resection device to remove the tissue slug produced. Optionally, a fluid source may be provided remote from the handle in communication with the pump mechanism. The tissue resecting system may further comprise a fluid reservoir in the handle in communication with the pump mechanism configured to temporarily hold fluid, and the pump mechanism may include a positive displacement pump. The inner sleeve is configured to move at least one of axially and rotationally.

In a third aspect of the present invention, a tissue resecting system includes a handle coupled to an elongated outer sleeve with a closed distal end and a window that opens to an interior lumen. An inner sleeve is adapted to move longitudinally in said lumen between a window open position and a window closed position to resect tissue in protruding through the window. A resilient element is disposed in distal end of said lumen adapted to interface with the distal end of the inner sleeve when in its distal-most position. The tissue resecting system may further comprising a flow channel within the outer sleeve with an open termination in or proximate to the resilient element. The resilient element typically has a surface feature that interfaces with the distal end of the inner sleeve when in its distal-most position to seal the distal end of the passageway in the inner sleeve.

In a fourth aspect of the present invention, tissue resecting system includes a handle coupled to an elongated outer sleeve with a closed distal end and having a window that opens to an interior lumen. A motor drives an inner sleeve to reciprocate longitudinally in a first distal direction to resect tissue which protrudes through the window and in a second proximal direction to open the window. The inner sleeve carries an electrode for applying RF energy to tissue, and a controller modulates RF energy application and applies first RF energy parameters to the electrode when the inner sleeve moves in the first direction and applies second RF energy parameters to the electrode when the inner sleeve moves in the second direction.

### BRIEF DESCRIPTION OF DRAWINGS

FIG. 1 is a side view of a tissue resecting probe and block diagram of systems and operating components corresponding to the invention.

FIG. 2 is a perspective view of the working end of the resecting probe of FIG. 1 showing the inner resecting sleeve in a proximal or retracted position providing a window-open position.

FIG. 3 is another perspective view of the working end of the resecting probe of FIG. 1 showing a resilient element that interfaces with the reciprocating inner sleeve at the distal end of its stroke.

FIG. 4A is a sectional view of the working end of FIG. 2 with the reciprocating resecting sleeve in a proximal position at the beginning of its stroke in a window-open position.

FIG. 4B is a sectional view of the working end as in FIG. 4A with the reciprocating resecting sleeve in a distal position at the end of its stroke in a window-closed position.

FIG. 5A is a sectional view of the shaft of the probe of FIG. 1 taken along line 5A-5A of FIG. 4A.

FIG. 5B is a sectional view of the shaft of the probe of FIG. 1 taken along line 5B-5B of FIG. 4A.

FIG. 6A is a longitudinal sectional view of the shaft and handle of the probe of FIG. 1 showing the motor and drive mechanism with the resecting sleeve at the beginning of its stroke in a window-open position.

FIG. 6B is an enlarged sectional view of the working end of FIG. 6A with the resecting sleeve at the beginning of its stroke.

FIG. 7A is a longitudinal sectional view of the shaft and handle as in FIG. 6A showing the motor and drive mechanism with the resecting sleeve at the end of its stroke in a window-closed position.

FIG. 7B is an enlarged sectional view of the working end of FIG. 7A with the resecting sleeve at the end of its stroke.

FIG. 8 is an enlarged sectional view of the distal portion of the window of the working end of FIGS. 2-3.

FIG. 9 is an illustration of a method of using a device similar to that of FIG. 1 in a tissue resection procedure in a patient's prostate, wherein the resection is initiated within the urethra.

FIG. 10 is an illustration of an alternative method of using the device of FIG. 1 in a tissue resection procedure in a patient's prostate wherein the distal end is penetrated into the lobe of the prostate.

### DETAILED DESCRIPTION OF THE INVENTION

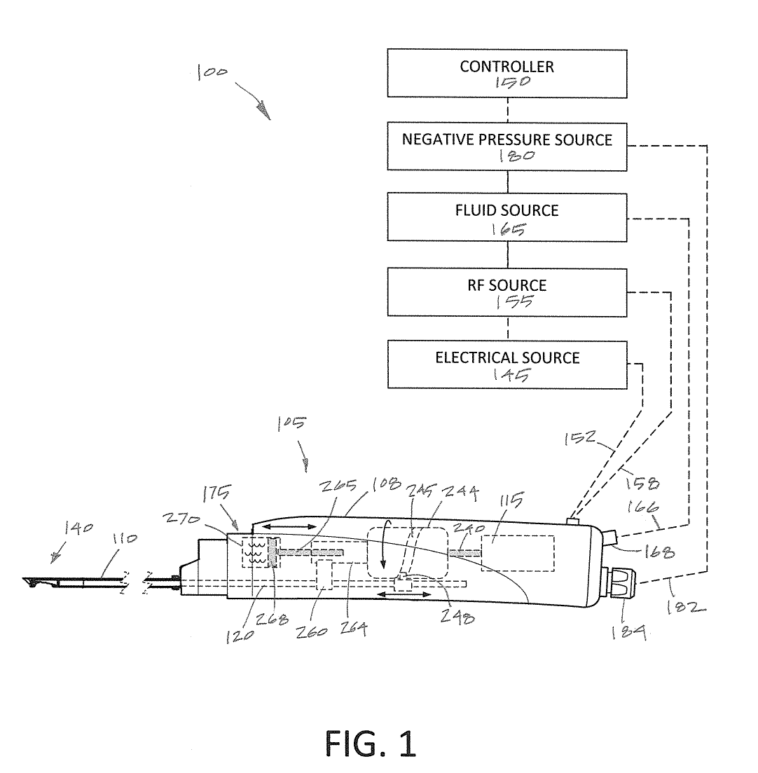

FIGS. 1-3 illustrate an electrosurgical tissue resecting system 100 that includes a hand-held single-use tissue resecting device or probe 105. The device 105 has a handle portion 108 that is coupled to an elongated shaft or extension portion 110 that has an outer diameter ranging from about 2 mm to 5 mm. In one variation, the device is adapted for performing a TURP procedure (transurethral resection of the prostate) and the shaft portion 110 extending about longitudinal axis 112 can have a length suitable for introducing though an endoscope or cystoscope to thereby access a male patient's prostate to resect and remove tissue.

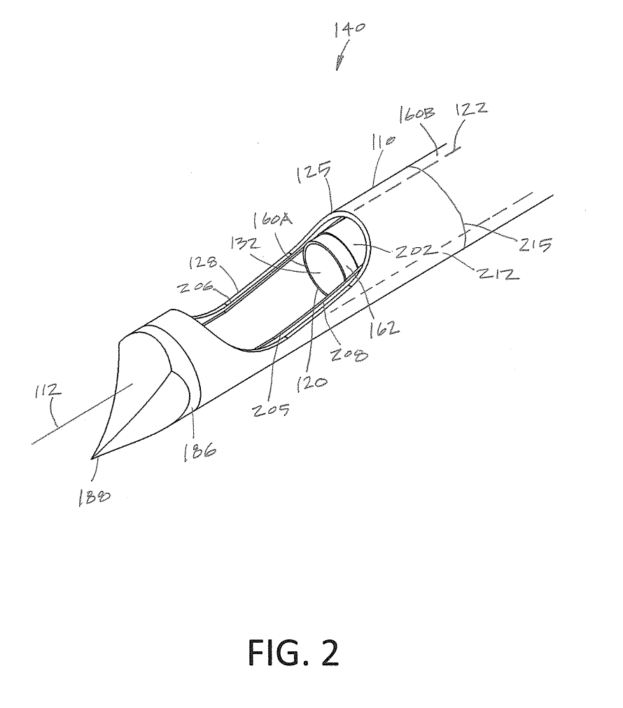

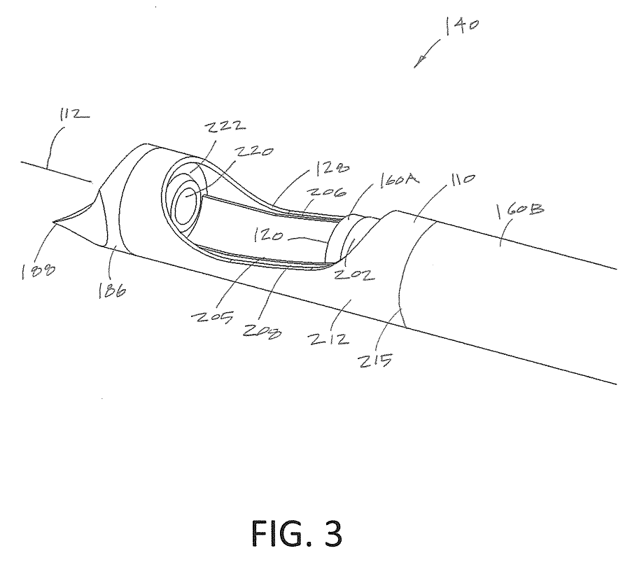

Referring to FIG. 1, it can be seen that the handle 108 carries an electric motor 115 for reciprocating or rotating a resecting element, which in the variation of FIG. 1-3 is an inner sleeve 120 that reciprocates in a passageway 122 defined within the interior of an outer sleeve 125 to resect tissue interfacing window 128 in the outer sleeve. Resected tissue masses, referred to herein as “slugs,” are captured in channel 132 of inner sleeve 120 and can be extracted or moved in the proximal direction in the channel by both positive and negative pressures on either side of the tissue slug as will be described below. FIGS. 2-3 illustrate the working end 140 of the resecting device 105 with the moveable inner resecting sleeve 120 in a retracted position relative to window 128. The motor 115 in handle 108 is operatively coupled to an electrical source 145 and controller 150 by electrical cable 152. The controller 125 can include algorithms adapted to control motor voltage which in turn can control the speed of reciprocation or rotation of the resecting sleeve 120 as will be described below.

Still referring to FIG. 1, the system 100 includes an RF source 155 operatively coupled by cable 158 to first and second opposing polarity electrodes 160A and 160B in the working end 140 (FIG. 2). The distal end 162 of inner sleeve 120 comprises a first or active electrode 160A which is adapted to form an electrosurgical plasma for resecting tissue as is known in the art. The second or return electrode 160B can comprise a medial portion of outer sleeve 125 and/or the distal tip component of the shaft portion 110.

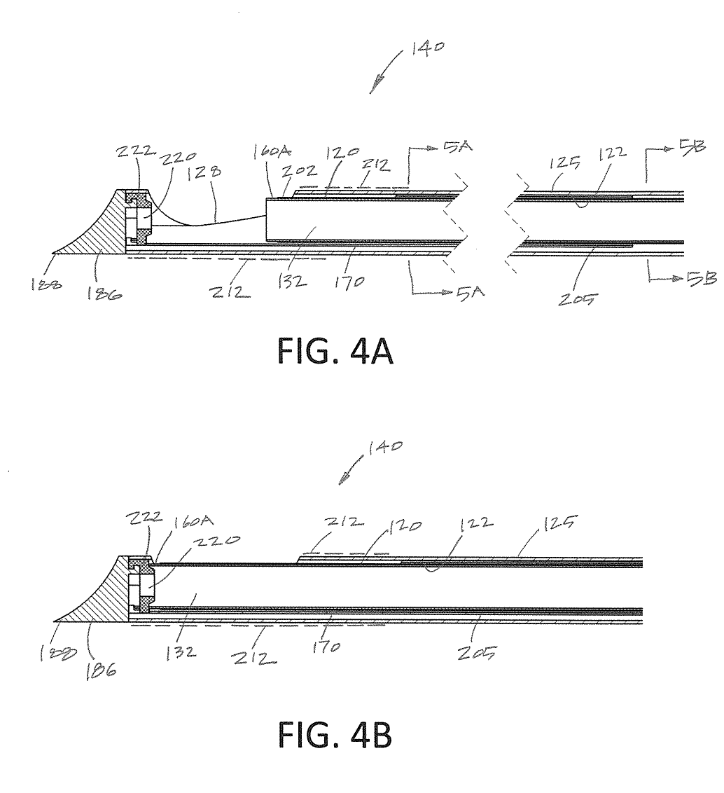

FIG. 1 further illustrates that the system 100 includes a remote fluid source 165 which has flow line 166 extending to a coupling 168 in handle 108. The fluid source 165 can comprise a bag of saline and flow line 166 is in fluid communication with a flow channel 170 in the shaft portion 110 to carry fluid through to handle 108 to the working end 140 by means of a pump mechanism 175 carried in the handle 108 (FIG. 4A).

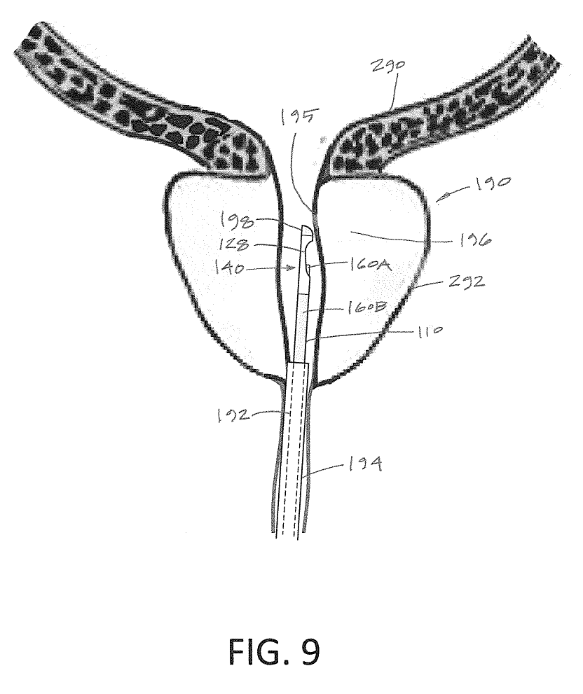

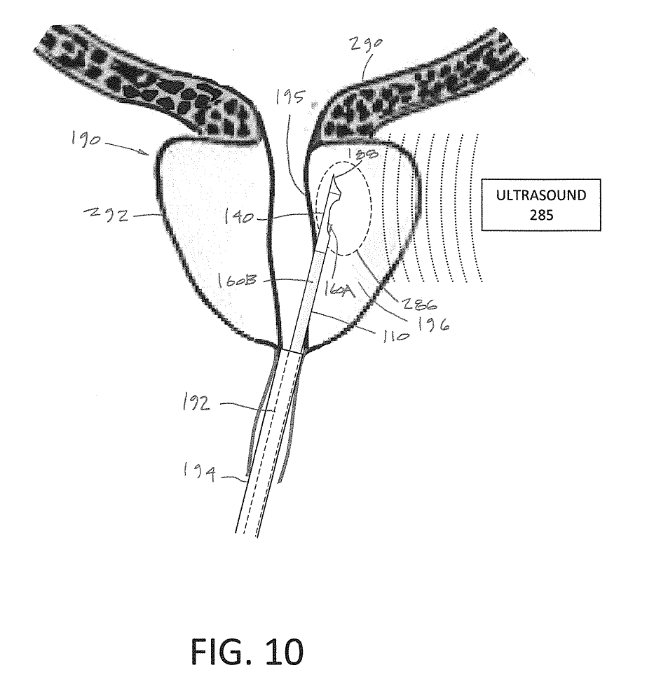

FIG. 1 also shows that the system 100 has a remote negative pressure source 180 that has suction line 182 that couples to fitting 184 in the handle 108. The negative pressure source 180 communicates with a tissue-extraction channel 132 in the resecting sleeve 120 for assisting in extracting tissue from the site of the resection, for example in the patient's prostate (see FIGS. 9-10).

As can be understood from FIG. 1, the controller 150 includes algorithms for operating and modulating the operating parameters of the subsystems, including the RF source 155, the fluid source 165 and associated pump mechanism 175, the negative pressure source 180 and the motor 115 via motor voltage. As will be described below, in some operating modes, the controller 150 may adjust operating parameters of all subsystems during each reciprocation cycle of the resecting sleeve 120 to achieve various operating objectives.

Now turning to FIGS. 4A-7B, the structure and operation of the working end 140 can be described. A similar device can be made with a sharp tip or a rounded tip for different approaches for prostate resection. In one variation shown in FIGS. 2-4B, it can be seen that the distal end body 186 with a sharp tip 188 of the device is sharp for penetrating tissue. In a resection procedure in a prostate 190 (see FIG. 10) such a sharp tip 188 is adapted for extending outward from the working channel 192 of an endoscope or cystoscope 194 to penetrate through the urethra 195 into a prostate lobe 196 under suitable imaging such as ultrasound. This method would spare the urethra 195 except for one or more puncture sites. In another embodiment shown in FIG. 9, the device can have a rounded distal tip 198 and the resection procedure can be started within the urethra 195 and progress outwardly into the prostate lobe 196 in a method similar to conventional TURP procedures which use an RF loop and which do not spare the urethra 195.

FIGS. 2, 3 and 4A-4B illustrate the movement of the resecting sleeve 120 and explain how the working end 140 is configured to resect tissue, for example in a prostate. Referring to FIGS. 2, 3 and 4A, the inner resecting sleeve 120 is typically fabricated of thin-wall stainless steel but any other suitable materials can be used. The inner sleeve 120 has a thin insulative coating 202 around it exterior except for a distal end portion 162 that comprises electrode 160A. The exposed distal portion 162 that comprises electrode 160A can have a length ranging from about 1 mm to 6 mm. In one variation, the inner sleeve has an OD of 0.106″ and an ID of 0.098″. The insulative coating is parylene, but other dielectric polymers or ceramic coating are possible, such as PFA, polytetrafluroethylene (PTFE), FEP (Fluorinated ethylenepropylene), polyethylene, polyamide, ECTFE (Ethylenechlorotrifluoro-ethylene), ETFE, PVDF, polyvinyl chloride or silicone. The proximal end of inner resecting sleeve 120 is coupled to the electrical cable 158 within handle 108 and to a first pole of the RF source 155 (FIG. 1).

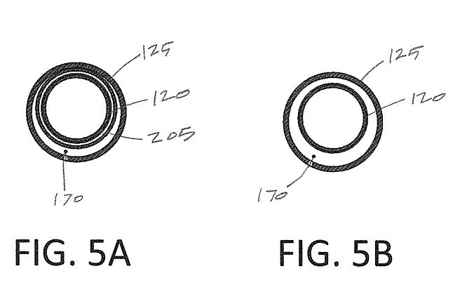

In FIGS. 2, 4A-4B and 5A-5B, it can be seen the outer sleeve 125 at the working end 140 comprises an assembly of outer sleeve 125 and thin wall intermediate sleeve 205 that when combined with outer sleeve 125 defines a flow channel 170 between the sleeves (see FIG. 5A). The intermediate sleeve 205 can extend to the handle 108 or can terminate in a medial region of shaft portion 110 as indicated in FIGS. 4A and 5B. Thus, it can be understood that inner sleeve 120 reciprocates in passageway 122 that is defined by intermediate sleeve 125. In FIGS. 2 and 3, it can be seen that the window 128 is defined by edges 206 and 208 of the outer sleeve 125 and intermediate sleeve 205, respectively, and the edges are welded or sealed by an insulative coating so that flow channel 170 has no open terminal portion around window 128. In FIGS. 4A-4B, a continuous parylene coating 212 (or other insulative coating) is provided about the exterior of outer sleeve and around the window 128 and in the passageway 122 in the intermediate sleeve 205. This coating 212 can extend proximally from the window from 5 mm to 50 mm to a terminal edge 215 (see FIGS. 2, 3 and 4A). Proximal to the edge 215 of the coating 212 on the outer sleeve is the exposed surface of outer sleeve 125 which comprises the return electrode 160B. The proximal end of inner resecting sleeve 120 is coupled to electrical cable 158 within handle 108 and thus to a second pole of the RF source 155.

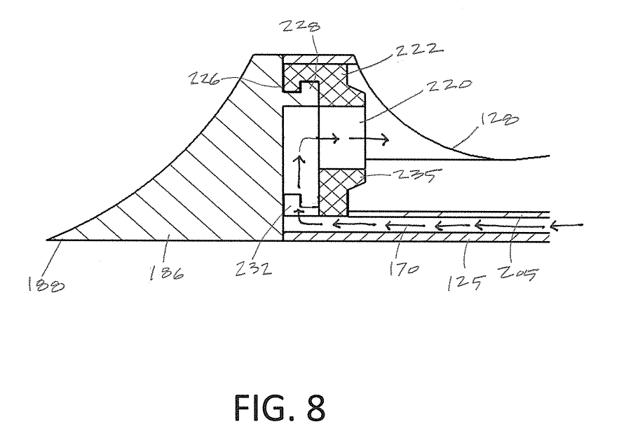

FIGS. 3, 4A-4B, and 8 illustrate that the flow channel 170 has a open termination 220 facing in the proximal direction in the center of dielectric element 222 that is fixed to distal end component 186. In one variation as shown in FIG. 8, the dielectric element 222 is of a resilient material such as silicone and with a circular edge 226 that fits over and grips metal edge 228 of the distal end component 186. In the enlarged view of FIG. 8, it can be seen that flow channel 170 transitions to gap 232 in the dielectric element 222 which allows fluid to flow from channel 170 through gap 232 and then in a reversed (proximal) direction through open termination 220. FIGS. 4A-4B and 8 show that dielectric element 222 has a circular ledge 235 around open termination 220 that is adapted to project slightly into and engage the electrode 160A and distal end 162 of inner sleeve 120 when this sleeve is at its distal-most position in each cycle of its reciprocation. The dielectric element 222 thus can form a seal with distal end 162 of the inner sleeve 120 at the distal-most position of its stroke. As will be described below, during a brief interval when inner sleeve 120 is at the end of its stroke and sealed against the dielectric element, then the pump mechanism 175 can provide a fluid flow burst through channel 170 and open termination 220 which causes resected tissue to be pushed proximally in tissue extraction channel 132 in inner sleeve 120.

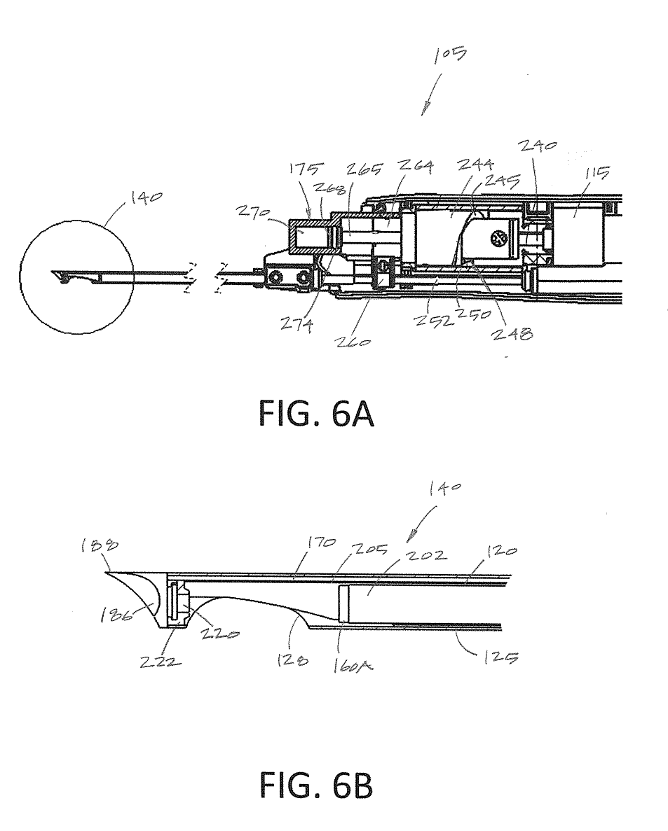

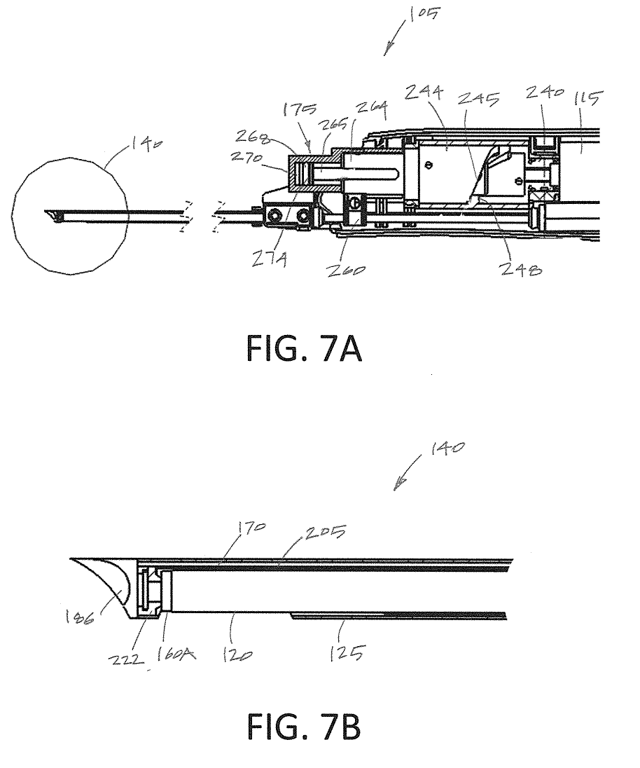

FIGS. 1, 6A and 7A show in general indicate how the single motor 115 in handle operates to both reciprocate the inner resecting sleeve 120 and the pump mechanism 175. In FIG. 6A, it can seen that motor 115 together with an internal gear reduction mechanism rotates shaft 240 which is coupled to a rotating drive collar 244 which converts rotary motion to axial motion. An arcuate or partly helical slot 245 in the drive collar 244 cooperates with a pin 248 in non-rotating body 250 that is keyed in the handle 108 and fixed to the proximal end 252 of the inner resecting sleeve 120. The motor 115 can comprise any suitable electrical motor, for example, a brushless electric motor. The motor 115 (and operation of other subsystems) can be actuated by a user-operated switch, which typically is a footswitch but also could be a handswitch. As can be understood from FIGS. 6A-7B, the rotation of drive collar 244 will cause non-rotating body 250 to reciprocate and cause the inner sleeve 120 to move between the window-open position of FIGS. 6A-6B and the window-closed position of FIGS. 7A-7B. In one variation, referring to FIGS. 6A-7B, the drive collar 244 can rotate 360° with a continuous arcuate slot 245 to thus move the inner resecting sleeve 120 in a mechanical manner both in the distal direction to resect tissue captured in the window and in the proximal direction to re-open the window. In another embodiment, the drive collar 244 can operate as a cam to move the inner sleeve in the distal direction while also loading a spring mechanism (not shown), and then the spring mechanism can move the inner sleeve in the proximal direction back to the window-open position.

The speed of motor 115 can be constant through a cycle of reciprocation at a rate between 1 Hz to 5 Hz, or the controller 150 can use an algorithm to alter motor voltage to cause the motor to move the inner sleeve forward (distal direction) to resect tissue at a first speed and then move backward (proximal direction) at a second speed. In one variation as further described below, the controller 150 can control the RF source 155 to provide a constant power level which is adapted to generate a plasma about electrode 160A for resecting tissue during the forward stroke and then the same plasma can be used on the backward stroke to coagulate the tissue surface. In this variation, the backward stroke can be slowed down to provide a longer interval in which electrode 160A contacts tissue to increase the depth of coagulation. In the variation just described, motor voltage was modulated to alter the speed of the inner sleeve. It should be appreciated that the drive sleeve can rotate at a constant rate and the arcuate slot 245 in drive collar 244 and the cooperating pin 248 can be designed to provide the inner sleeve 120 with different effective forward and backward speeds. This would achieve the same result as modulating motor voltage to alter reciprocating speed.

FIGS. 1, 6A and 6B also illustrate how the motor 115 in handle 108 actuates the pump mechanism 175. In FIG. 6A, it again can seen that motor 115 rotates the drive collar 244 which in turn engaged pin 248 and causes the non-rotating body 250 to reciprocate. FIG. 6A also shows an actuator 260 fixed to the inner sleeve 120 within handle 108 that reciprocates to drive the pump mechanism 175. Referring to FIGS. 6A and 7A, the actuator 260 extends laterally and is coupled to cylindrical element 264 that also reciprocates in the handle 108, with part of its stroke extending into a bore in the drive collar 244. The cylindrical element 264 is coupled to shaft 265 of a piston 268 that moves back and forth in pump chamber 270 to thereby pump fluid from fluid source 165 through channel 170 (FIGS. 4A-5B) to the working end 140. It can be seen that piston 268 has o-rings 274 to seal the pump chamber 270 and fluid can be delivered to the pump mechanism through flow line 166 (FIG. 1) by gravity or other suitable means such as a pump. The fluid flows into and out of the pump chamber 270 can be facilitated by one-way valves are known in the art.

In the variation shown in FIGS. 6A-7B, the pump system 175 is actuated by the drive collar 244 which operates the resecting sleeve 120, and thus the reciprocation rate and/or the varied speed of the piston shaft 256 will match that of the resecting sleeve. The flow rate of fluid through the system will then be determined by the selected speed profile of the inner sleeve's reciprocation. In another variation, it is preferable to have a pulse of fluid flow through channel 170 and open termination 220 within a very brief time interval when then inner sleeve 120 is at the end of its stroke and sealed against the dielectric element 222. In this variation, the pulse of liquid can range from 1 cc to about 5 cc and is adapted to push a slug of resected tissue under positive pressure in the proximal direction in the tissue extraction channel 132. In order to provide such a pulsed flow, the drive collar 244 can have a second arcuate slot to drive a second pin (not shown) to drive the piston shaft 265 with any selected interval. In one variation, the pulse of fluid flow occurs within less than 0.2 seconds or less than 0.1 seconds. In another embodiment, the drive collar 244 can operate both the resecting sleeve 120 and the piston shaft 265 in unison and the fluid can flow into an intermediate reservoir in handle 108 (not shown) which can be configured to release the fluid into channel 170 only within a selected time interval.

FIG. 9 illustrates a step in a method of using the device 100 of FIG. 1 to resect tissue in a patient's prostate. In the method of FIG. 9, the physician introduces a cystoscope 194 transurethrally to view the prostate. The shaft 110 of a rounded end probe 100 is advanced through the working channel 192 of the cystoscope and after viewing appropriate landmarks as known in the art, the device is actuated to resect and extract tissue. In this method, a saline irrigation fluid is controllably delivered by a pump (or gravity flow) to the site through another channel in the cystoscope to immerse the treatment site in saline. In operation, the negative pressure source 180 can be actuated continuously, which communicates with tissue extraction channel 132 in the inner sleeve and functions to draw tissue into window 128. At the same time, the negative pressure source 180 will remove saline from site when the window is open to thus cause circulation of fluid through the treatment site, which will remove blood and debris to keep the saline clean for enhancing endoscopic viewing of the resection procedure. The saline inflows can be managed by any conventional fluid management system as is known in the art, wherein such systems include pressure sensing or pressure calculation mechanisms for monitoring and controlling fluid pressure in the treatment site. The working end 140 then can be moved axially and rotationally under direct endoscopic vision while the resecting sleeve 120 is actuated to resect and extract tissue. As described above, the actuation of a switch, such as a foot pedal will cause the controller to actuate (i) reciprocation of the resection sleeve 120, (ii) the pump mechanism 175, (iii) the negative pressure source 180, and the RF source 155. In one variation, the RF source 155 is controlled to operate continuously at a power level that created a plasma about electrode 160A to resect tissue as the sleeve 120 moves in the distal direction across window 128. In this variation, the plasma about electrode 160A coagulates tissue in contact with the electrode 160A as it moves in the proximal direction. The resected tissue slugs are captured in a collection container (not shown). In other methods of coagulating tissue in TURP procedure shown in FIG. 9, (i) the resecting sleeve can be moved in the proximal direction at a slower speed to allow the plasma about electrode 160 to be in contact with tissue for a longer interval, (ii) the controller can switch the output and/or power from RF source 155 to a different parameter for better coagulation on the return stroke of the resecting sleeve 120, and/or (iii) the resecting sleeve 120 can be stopped in a selected position within window 128 and the RF output and power can be delivered between electrodes 160A and 160B to allow the physician to manipulate the working end to coagulate targeted tissue. In the event that the system is operated in different modes, for example a plasma resection mode and a coagulation mode, then a conventional electrosurgical foot pedal system may be used with a first pedal for tissue resection and a second pedal for coagulation.

FIG. 10 illustrates another method of using device 100 of FIG. 1 to resect tissue in a patient's prostate. In the method of FIG. 10, the physician again introduces a cystoscope 194 transurethrally into a patient's prostate, and then advances shaft 110 of a sharp-tipped probe 100 through the working channel 192 of the cystoscope and thereafter through the urethra 195 into the prostate lobe 196. In this method, FIG. 10 shows that the tissue resection is done interstitially and thus viewing is needed which can be provided by ultrasound system 285 or another suitable type of imaging. In one variation, the ultrasound system is a TRUS system as known in the art. A conventional fluid management system can be used as described above to irrigate and maintain fluid pressure in the urethra 195. In FIG. 10, a target of the treatment is to resect and extract tissue region 286 wherein fluid from within the urethra 195 may not flow into the cavity being resected in the prostate lobe and for this reason fluid flow from the working end 140 can fill the space around the working end 140. In one variation, the pump mechanism 175 operates during the reciprocation cycle and saline will flow through channel 170 and outward from open termination 220 and outward from window 128 into the resected cavity, except for when the window 128 is closed. The saline thus irrigates the space around the working end 140 and supports the RF plasma formation about electrode 160A. In this method, the fluid flow volume through channel 170 can be set at any selected volume per cycle of reciprocation, for example from 0.5 cc to 10 cc's. This selected volume can be unrelated to an optional pulsed volume when the window 128 is closed. The volumes of fluid pumped can be adjusted by design of the volume of the pump chamber and piston stroke. In another embodiment, the probe shaft 110 could be configured with another flow channel to deliver fluid to the space in the prostate lobe 196 around the working end 140.

Still referring to the resection method of FIG. 10, the working end 140 also can be operated in another manner to cause coagulation. As described above, the resecting sleeve 120 can be stopped in a selected position within window 128 and optimal RF output and power can be delivered between electrodes 160A and 160B to heat fluid in the space around the working end 140 which will effectively coagulate tissue around the resected cavity. In such a coagulation mode, a foot pedal can be used to activate the system, and in on variation the foot pedal could be tapped to cause the coagulation mode to operate for a predetermined time interval, for example 10 seconds, 20 seconds, 20 seconds or 60 seconds. In another variation, the foot pedal could be depressed to actuate the coagulation mode until the pedal is released.

While the above embodiments have described a system that has a single motor 115 that operates both the resecting sleeve 120 and the pump mechanism 175, another variation could have a first motor in handle 108 that operates the resecting sleeve 120 and a second motor that actuates the pump mechanism 175. This option would allow the controller 150 to independently modulate parameters of both systems during each cycle of reciprocation and thus potentially allow for more modes of operation

In the embodiment of FIGS. 1-3, the distal end of the probe has a fixed sharp tip 188. In another embodiment, a sharp tipped needle (not shown) could be extended and retracted from the distal body 186 by manipulation of an actuator portion in handle 108. In this variation, the needle tip would be extended only when the physician penetrated the working end through the urethra 195 (cf. FIG. 10). Thus, during the steps of resecting tissue in the prostate lobe under imaging, the dull tip of the probe would make it impossible or unlikely that the physician could inadvertently push the tip into the bladder 290 or through the prostate capsule wall.

In general, the tissue resecting device corresponding to the invention comprises a handle and elongated sleeve assembly comprising a windowed outer sleeve and an inner sleeve adapted to move relative to the window to resect tissue, and a motor in the handle configured to move the inner sleeve and operate a pump to provide a fluid flow through a channel in the sleeve assembly. In one variation, the tissue resecting has an inner resecting sleeve that is adapted to reciprocate relative to the window. In another embodiment, the resecting sleeve is adapted to rotate relative to the window. In another embodiment, the resecting sleeve is adapted to reciprocate and rotate relative to the window.

In another aspect of the invention, the pump mechanism 175 of FIGS. 1, 6A and 7A is a positive displacement pump and more specifically a piston pump. In other variations, the pump can be is selected from the group consisting of piston pumps, screw pumps, impeller pumps, peristaltic pumps, vane pumps, lobe pumps, plunger pumps and diaphragm pumps.

In another aspect of the invention, the resecting sleeve 120 comprises an electrode for electrosurgically resecting tissue. In another variation, the resecting sleeve can have a blade edge for cutting tissue.

In another aspect of the invention, the tissue resecting system includes a probe with an elongated sleeve assembly comprising a windowed outer sleeve and an inner sleeve adapted to move in a cycle to resect tissue interfacing with the window, and a pump mechanism in or proximate the handle configured to provide a fluid flow through a channel in the sleeve assembly. The pump mechanism can be adapted to provide the flow at a constant rate over each cycle of the inner sleeve, or the pump mechanism can be adapted to provide the flow at a non-constant rate over each cycle of the inner sleeve. In one variation, the pump is operated to provide a pulsed fluid flow.

In another aspect of the invention, the tissue resecting system includes a probe having an elongated outer sleeve with a closed distal end with a side-facing window that opens to an interior lumen in the sleeve, with an inner sleeve adapted to move longitudinally in the lumen between window open and window closed positions to thereby resect tissue in the window, and a resilient element disposed in distal end of the lumen adapted to interface with the distal end of the inner sleeve when in its distal-most position. In this variation, the system also includes a flow channel within the outer sleeve having an open termination in or proximate to the resilient element, wherein the resilient element is configured to contact the inner sleeve in its distal-most position to seal the distal end of the passageway in the inner sleeve.

In another aspect of the invention, the tissue resecting system includes a probe having an elongated outer sleeve with a closed distal end and side-facing window that opens to an interior lumen, a motor driven inner sleeve adapted to reciprocate longitudinally in a first distal direction across the window to resect tissue and in a second proximal direction to thereby open the window wherein the inner sleeve carries an electrode for applying RF energy to tissue and wherein a controller moves the inner sleeve in the first direction at a first speed and moves the inner sleeve in the second direction at a second different speed. Different RF parameters can be used in the first and second directions.

1. (canceled)

2. A tissue resecting system comprising:
a handle;
an elongated sleeve assembly configured to be coupled to the handle, said elongated sleeve assembly comprising a windowed outer sleeve and an inner sleeve adapted to move in a cycle to resect tissue interfacing with the window, said elongated sleeve assembly having a capture channel configured to receive tissue resected by the inner and outer sleeves and an flow channel defined between an outer surface of the inner sleeve and an inner surface of the outer sleeve;
a pump mechanism in or proximate the handle configured to deliver a fluid flow into a proximal end of the flow channel to a working end of the elongated sleeve to remove resected tissue from the capture channel.

3. The tissue resecting system of claim 2 wherein the pump mechanism is adapted to provide the flow at a constant rate over each cycle of the inner sleeve.

4. The tissue resecting system of claim 2 wherein the pump mechanism is adapted to provide the flow at a non-constant rate over each cycle of the inner sleeve.

5. The tissue resecting system of claim 2 wherein the pump mechanism is adapted to provide the flow in at least one pulse.

6. The tissue resecting system of claim 2 wherein the pump mechanism is adapted to provide the flow when the inner sleeve is in a position that closes the window in the outer sleeve.

7. The tissue resecting system of claim 2 wherein a flow volume ranges between 1 cc and 10 cc during each cycle.

8. The tissue resecting system of claim 2 further comprising a fluid source remote from the handle in communication with the pump mechanism.

9. The tissue resecting system of claim 8 further comprising a fluid reservoir in the handle in communication with the pump mechanism configured to temporarily hold fluid.

10. The tissue resecting system of claim 2 the pump mechanism includes a positive displacement pump.

11. The tissue resecting system of claim 2 wherein the inner sleeve moves at least one of axially and rotationally.
